(19) United States
(12) Patent Application Publication
(10) Pub. No.: US 2024/0299661 A1
(43) Pub. Date: Sep. 12, 2024

(54) ELECTRO-PNEUMATIC INTELLIGENT SYRINGE

(71) Applicant: Huzhou University, Huzhou (CN)

(72) Inventors: Yue GUO, Huzhou (CN); Zhen Wang, Huzhou (CN); Hongchang Zhou, Huzhou (CN); Juling Xu, Huzhou (CN); Lei Zhao, Huzhou (CN); Limin Liu, Huzhou (CN)

(21) Appl. No.: 18/472,266

(22) Filed: Sep. 22, 2023

(30) Foreign Application Priority Data
Mar. 10, 2023 (CN) 2023102570530

(51) Int. Cl. A61M 5/20; A61M 5/31

(52) U.S. Cl. CPC A61M 5/20; A61M 5/3148

(57) ABSTRACT

An electro-pneumatic intelligent syringe includes an injection assembly including a fixing component and a moving component, and a pneumatic assembly including an airway component and a pneumatic component. The electro-pneumatic intelligent syringe can operate intelligently and automatically, which provides equipment support for achieving automatic injection. The electro-pneumatic intelligent syringe can automatically complete the medicine suction and injection operations of the syringe without human control, has high degree of automation, unmanned isolation operation, strong intelligence, safety and efficiency, convenient use, and easy popularization, can avoid direct contact between doctors and patients during the injection process, can effectively reduce the possibility of medical staff being infected by viruses, and is conducive to protecting relevant medical staff, especially suitable for epidemic prevention and epidemic resistance of highly infectious diseases such as corona virus disease 2019 (COVID-19) vaccination and other occasions.

### TECHNICAL FIELD

The disclosure relates to the technical field of medical equipment, and particularly to an electro-pneumatic intelligent syringe.

### BACKGROUND

At present, due to the influence of corona virus disease 2019 (COVID-19), the task of epidemic prevention and fighting is heavy, and the workload of injection is increased. However, the current automatic injection equipments have the following shortcomings:
1. No implementation plan for automatic injection has been found.
2. Lack of solutions for implementing intelligent medicine suction processing equipment for automatic syringes.
3. Lack of an executable solution for unmanned automatic completion of syringe suction.

### SUMMARY

The disclosure provides an electro-pneumatic intelligent syringe to solve the problems in the prior art.

The electro-pneumatic intelligent syringe includes an injection assembly and a pneumatic assembly. The injection assembly includes a fixing component and a moving component, and the pneumatic assembly includes an airway component and a pneumatic component. The fixing component includes a syringe needle and a needle tube. And a bottom of the syringe needle is fixed to the needle tube, the syringe needle defines a hole penetrating through itself and the hole of the syringe needle is connected with a needle through hole defined on the needle tube, and an interlayer is provided between the needle tube and the syringe needle. The moving component includes a syringe plunger, a sealing stack embedded into a middle of the syringe plunger, and a sealing pressure rod provided on a side of the sealing stack facing towards the pneumatic assembly. The airway component includes a central airway, a plunger airway, and a pressure rod airway, and the central airway, the plunger airway and the pressure rod airway each extend along with a moving of the syringe plunger. The pneumatic component includes a suction-supply fan, a multi-way valve, a component framework, and a controller; and the suction-supply fan is connected to a fan mouth of the multi-way valve.

In an embodiment, a needle sealing door and an induction coil are disposed in the interlayer, and the needle sealing door is disposed on the needle through hole.

In an embodiment, a side of the sealing stack is connected with the needle tube, and another side of the sealing stack is fixedly connected with the central airway.

In an embodiment, the sealing pressure rod includes a pressure rod arm and an air-controlled pressure rod shaft, and the air-controlled pressure rod shaft is fixedly connected to the pressure rod airway.

In an embodiment, a side of the central airway is connected to a center mouth of the multi-way valve, another side of the central airway is connected to the sealing stack; a side of the plunger airway is connected to a plunger mouth of the multi-way valve, another side of the plunger airway is connected to the syringe plunger; a side of the pressure rod airway is connected to a pressure rod mouth of the multi-way valve, and another side of the pressure rod airway is connected to the sealing pressure rod.

In an embodiment, the controller is a system on a chip (SoC) microcontroller including a built-in memory, a central processing unit (CPU), interfaces detection and driving circuits, a wireless communication module, a wireless charging circuit, a BLUETOOTH module.

In an embodiment, a material of the component framework is a plastic frame with high-strength and non-toxic.

The technical effects of the disclosure are as follows.

The electro-pneumatic intelligent syringe can automatically complete the treatment of medicine suction and injection of the syringe without human control, has high degree of automation, unmanned isolation operation, strong intelligence, safety and efficiency, convenient use and easy popularization, can avoid direct contact between doctors and patients during the injection process, can effectively reduce the possibility of medical staff being infected by viruses, and is conducive to protecting relevant medical staff, especially suitable for epidemic prevention and epidemic resistance of highly infectious diseases such as COVID-19 vaccination and other occasions.

### BRIEF DESCRIPTION OF DRAWINGS

The accompanying drawings forming a part of the disclosure are used to provide a further understanding of the disclosure. The illustrative embodiments and explanations of the disclosure are used to explain the disclosure and do not constitute an improper limitation of the disclosure.

FIG. 1 is a schematic diagram of an overall structure of an electro-pneumatic intelligent syringe.

FIG. 2 is a schematic structural diagram of a pneumatic assembly of the electro-pneumatic intelligent syringe.

FIG. 3 is a schematic structural diagram of a sealing stack of the electro-pneumatic intelligent syringe.

FIG. 4 is a schematic structural diagram of a needle sealing door of the electro-pneumatic intelligent syringe.

Description of reference numerals: 1. syringe needle; 2. needle tube; 3. needle sealing door; 4. induction coil; 5. needle through hole; 6. syringe plunger; 7. power converter; 8. sealing stack; 9. sealing pressure rod; 10. plunger airway; 11. central airway; 12. electromagnetic push rod; 13. multi-way valve; 14. plunger mouth; 15. pressure rod airway 16. pressure rod mouth; 17. suction-supply fan; 18. component framework; 19. controller; 20. fan mouth; 21. center mouth; 22. pressure rod arm; 23. air-controlled pressure rod shaft.

### DETAILED DESCRIPTION OF EMBODIMENTS

It should be noted that in the absence of conflicts, the embodiments and the features in the embodiments of the disclosure can be combined with each other. The following will refer to the attached drawings and combine with embodiments to provide a detailed explanation of the disclosure.

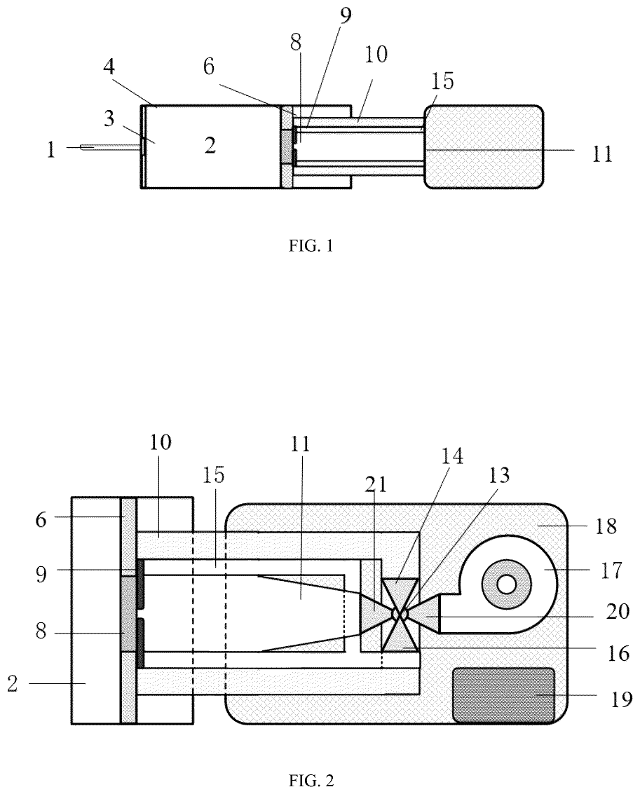

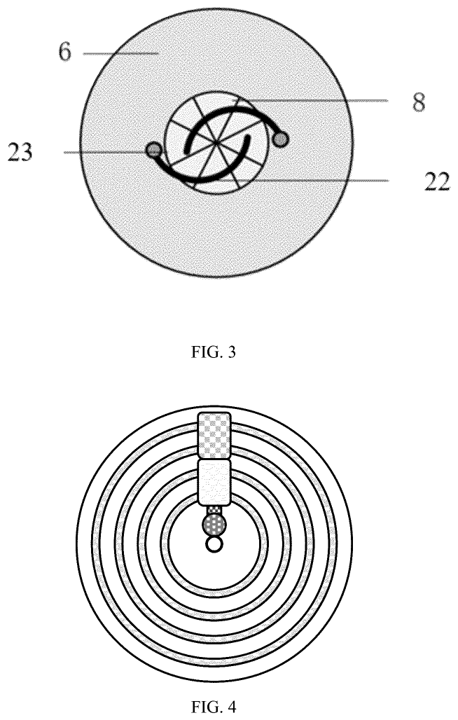

As shown in FIGS. 1-4, an electro-pneumatic intelligent syringe is provided.

The electro-pneumatic intelligent syringe includes an injection assembly and a pneumatic assembly. The injection assembly includes a fixing component and a moving component, and the pneumatic assembly includes an airway component and a pneumatic component.

The fixing component mainly includes a syringe needle 1, a needle tube 2, a needle sealing door 3 and an induction coil 4, which are used to complete a suction, containment, or an injection of a medicine solution. The syringe needle 1 is a hollow needle made of stainless steel, a bottom of the syringe needle 1 is fixedly connected to the needle tube 2, and the syringe needle 1 defines a hole penetrating through itself and the hole of the syringe needle 1 is connected with a needle through hole 5 defined on the needle tube 2. The needle tube 2 is a wear-resistant, high-strength and non-toxic plastic tube, an end of the needle tube 2 is fixedly connected to the syringe needle 1, the medicine solution can be sucked or discharged through the needle through hole 5 in an interlayer and the hole of the syringe needle 1. The needle sealing door 3 is a BLUETOOTH controlled electromagnetic sealing door with micro low-power, the BLUETOOTH controlled electromagnetic sealing door is composed of a micro waterproof direct current (DC) power electromagnetic push rod referred to as the electromagnetic push rod 12, and a micro sealing door made of a high-strength, non-toxic and wear-resistant plastic. In an initial or unoperated state, the needle sealing door 3 is automatically closed to block the needle through hole 5 between the syringe needle 1 and the needle tube 2. Under the control of a BLUETOOTH signal of a controller 19, the electromagnetic push rod 12 can drive the needle sealing door 3 to open the needle through hole 5, thus, the needle sealing door 3 is controlled by the BLUETOOTH signal of the controller 19. The induction coil 4 is a wireless power electromagnetic induction coil with an alternating current and direct current (AC/DC) power converter 7, which can supply power to the needle sealing door 3.

The moving component includes a syringe plunger 6, a sealing stack 8, a sealing pressure rod 9, which are all embedded in the needle tube 2, the syringe plunger 6 can move inside the needle tube 2, and providing power for the needle tube 2 to suction or discharge the medicine solution. The syringe plunger 6 is a wear-resistant, high-strength, non-toxic plastic hollow disc that is fixedly connected to a plunger airway 10 and can move back and forth in the needle tube 2 under the drive of the airflow in the plunger airway 10. A middle of the syringe plunger 6 is embedded with the sealing stack 8, and the sealing pressure rod 9 is provided on a side of the sealing stack 8 facing towards the pneumatic assembly. The sealing stack 8 is a high-strength, high toughness, wear-resistant, and non-toxic plastic sheet group coated with a wear-resistant sealing flexible coating on its surface, and the sealing stack 8 is a unidirectional opening and self-closing stacking group, with a side connected to the needle tube 2 and another side fixedly connected to a central airway 11. In a natural state, the sealing stack 8 is interlocked and self-sealing, under the action of a suction airflow in a central airway 11, the sealing stack 8 can open towards a direction of the pneumatic component, after the suction airflow disappears, the self-sealing of the sealing stack 8 will automatically resume. The sealing pressure rod 9 is a micro pneumatic curved high-strength and non-toxic plastic rod fixed on a wall of the plunger, which includes a pressure rod arm 22 and an air-controlled pressure rod shaft 23, and the air-controlled pressure rod shaft 22 is fixedly connected to a pressure rod airway 15. In a state of no external force effect, the pressure rod arm 22 is buckled on the sealing stack 8, so that the sealing stack 8 cannot be opened. When the air flow in the pressure rod airway 15 is operated under negative pressure on the air-controlled pressure rod shaft 23, the air-controlled pressure rod shaft 23 drives the pressure rod arm 22 to open, and the sealing stack 8 is in a state that can be opened. When the pressure rod airway 15 release the air, and the air-controlled pressure rod shaft 23 return to normal pressure, then the air-controlled pressure rod shaft 23 drives the pressure rod arm 22 to automatically reset, and the pressure rod arm 22 buckles on the sealing stack 8, so that the sealing stack 8 cannot be opened.

The airway component mainly includes a central airway 11, a plunger airway 10, and a pressure rod airway 15, which are all composed of non-toxic silica gel foldable tubes with high-strength, wear-resistance, high toughness, and high elasticity. In a natural state, the central airway 11, the plunger airway 10, and the pressure rod airway 15 are contraction and can be extended as needed with the movement of the plunger to transport and control the airflow of the moving component. The central airway 11 is connected to a center mouth 21 of the multi-way valve 13 and the sealing stack 8 of the moving component. The central airway 11 is an airflow channel that opens the sealing stack 8 through suction airflow and implements negative pressure operations on the needle tube 2. A side of the plunger airway 10 is connected to a plunger mouth 14 of the multi-way valve 13, another side of the plunger airway 10 is connected to the syringe plunger 6. The plunger airway 10 is an airflow channel that causes the plunger to move back and forth within the needle tube 2 by suctioning and delivering the airflow. A side of the pressure rod airway 15 is connected to a pressure rod mouth 16 of the multi-way valve 13, and another side of the pressure rod airway 15 is connected to the sealing pressure rod 9. The pressure rod airway 15 generates the negative pressure through suction, causing the air-controlled pressure rod shaft 23 to drive the pressure rod arm 22 to open the airflow channels.

The pneumatic component mainly includes a suction-supply fan 17, a multi-way valve 13, a component framework 18 and the controller 19, which are used to drive, control, and operate other components. The suction-supply fan 17 is a micro high-efficiency electrically controlled shockproof and waterproof suction-supply fan, the suction-supply fan 17 is connected to a fan mouth 20 of the multi-way valve 13, which can perform suction and supply operations. It is an important source of power for the device and provides pneumatic support for other components. The multi-way valve 13 is a microelectronic controlled one-way multi-way solenoid valve, with the fan mouth 20 as its input mouth, the center mouth 21, the plunger mouth 14, and the pressure rod mouth 16 as its output mouths. The input mouth can be controlled to select any output mouth, but only one output mouth can be selected at the same time. The multi-way valve 13 can close any output mouth, and the closed output mouth is in a closed state, neither inhaling nor delivering air. The function of multi-way valve 13 is to switch and connect one pneumatic source with three demand ends. The component framework 18 is a high-strength and non-toxic plastic framework used to fix, support, and install various equipment of the components.

The use and working process of the electro-pneumatic intelligent syringe are as follows:

1. The controller 19 communicates wirelessly with a medical of cloud center of the medical Internet of things (IOT), and receives relevant instructions and preset parameters. Under the command control of the controller 19, each component of the electro-pneumatic intelligent syringe enters the initial state, and the syringe plunger 6 retracts to the bottom of the needle tube 2; and the electro-pneumatic intelligent syringe prepares operations.

2. The sealing pressure rod 9 is opened. The controller 19 instructs the fan mouth 20 of the multi-way valve 13 to connect with the pressure rod mouth 16, and the suction-supply fan 17 starts the suction operation, causing negative pressure to form in the pressure rod airway 15, and the negative pressure makes the pneumatic pressure rod shaft 23 drive the pressure rod arm 22 to open, thus the sealing stack 8 is in an openable state. The pressure rod mouth 16 of the multi-way valve 13 is closed, negative pressure is maintained in the pressure rod airway 15, and the sealing pressure rod 9 continues to open.

3. Negative pressure operation of the needle tube. The controller 19 instructs the fan mouth 20 of the multi-way valve 13 to connect to the center mouth 21, causing the airflow in the center airway 11 to generate suction and form the negative pressure, and the sealing stack 8 is opened. The sealing stack 8 is open to make the needle tube 2 connected to the central airway 11, and then the needle tube 2 is generated suction to form the negative pressure. When the center mouth 21 of the multi-way valve 13 is closed, the sealing stack 8 is closed automatically. And the suction-supply fan 17 is shut down, and the fan mouth 20 of the multi-way valve 13 is connected to the pressure rod mouth 16. The negative pressure in the pressure rod airway 15 is relieved, and the sealing pressure rod 9 automatically resets to buckling the sealing stack 8.

4. Absorption operation. Both ends of the needle tube 2 are sealed by the needle sealing door 3 and the sealing stack 8 correspondingly, and the negative pressure inside needle tube 2 continues to be maintained. Then the fan mouth 20 of the multi-way valve 13 is connected to the center mouth 21 to relieve the negative pressure inside the center airway 11. After the negative pressure inside the center airway 11 is relieved, the syringe needle 1 is inserted into a bottle with the medicine solution, then the controller 19 instructs the needle sealing door 3 to open, and the negative pressure in the needle tube 2 make the medicine solution flow into the needle tube 2 from the bottle until the medicine solution in the bottle is suctioned completely. After the medicine solution is suctioned completely, the needle sealing door 3 is closed, and the medicine solution is maintained in the needle tube 2.

5. Injection operation. The controller 19 receives a signal for needle 1 to penetrate the skin and instructs the needle sealing door 3 to open, the fan mouth 20 of the multi-way valve 13 is connected with the plunger mouth 14. The suction-supply fan 17 starts the air supply operation, and conveying airflow can flow into the plunger airway 10 through the fan mouth 20 of the multi-way valve 13 and the plunger mouth 14, then the conveying airflow in the plunger airway 10 pushes the syringe plunger 6 forward until the syringe plunger 6 is pushed down to the top of the needle tube 2, followed by discharging all the medicine solution. And then the needle sealing door 3 is closed.

6. All components restore the initial states, restore the positions, and complete the operations. And the controller 19 uploads the relevant data to the medical IOT cloud center.

The above is only the preferred specific embodiment of the disclosure, but the scope of protection of the disclosure is not limited to this. Any change or replacement that can be easily thought of by those skilled in the art within the technical scope disclosed in the disclosure should be covered by the disclosure. Therefore, the protection scope of the disclosure should be based on the protection scope of the claims.

What is claimed is:

1. An electro-pneumatic intelligent syringe, comprising:
an injection assembly, wherein the injection assembly comprises a fixing component and a moving component; and
a pneumatic assembly, wherein the pneumatic assembly comprises an airway component and a pneumatic component;
wherein the fixing component comprises: a syringe needle (1) and a needle tube (2);
wherein a bottom of the syringe needle (1) is fixedly connected to the needle tube (2), the syringe needle (1) defines a hole penetrating through itself and the hole of the syringe needle (1) is connected with a needle through hole (5) defined on the needle tube (2), and an interlayer is provided between the needle tube (2) and the syringe needle (1);
wherein the moving component comprises a syringe plunger (6), a sealing stack (8) embedded into a middle of the syringe plunger (6), and a sealing pressure rod (9) provided on a side of the sealing stack (8) facing towards the pneumatic assembly;
wherein the airway component comprises: a central airway (11), a plunger airway (10), and a pressure rod airway (15); wherein the central airway (11), the plunger airway (10) and the pressure rod airway (15) each extend along with a moving of the syringe plunger (6); and
wherein the pneumatic component comprises: a suction-supply fan (17), a multi-way valve (13), a component framework (18), and a controller (19); wherein the suction-supply fan (17) is connected to a fan mouth (20) of the multi-way valve (13).

2. The electro-pneumatic intelligent syringe as claimed in claim 1, wherein a needle sealing door (3) and an induction coil (4) are disposed in the interlayer, and the needle sealing door (3) is disposed on the needle through hole (5).

3. The electro-pneumatic intelligent syringe as claimed in claim 1, wherein a side of the sealing stack (8) is connected with the needle tube (2), and another side of the sealing stack (8) is fixedly connected with the central airway (11).

4. The electro-pneumatic intelligent syringe as claimed in claim 1, wherein the sealing pressure rod (9) comprises a pressure rod arm (22) and an air-controlled pressure rod shaft (23), and the air-controlled pressure rod shaft (14) is fixedly connected to the pressure rod airway (15).

5. The electro-pneumatic intelligent syringe as claimed in claim 1, wherein a side of the central airway (11) is connected to a center mouth (21) of the multi-way valve (13), another side of the central airway (11) is connected to the sealing stack (8); a side of the plunger airway (10) is connected to a plunger mouth (14) of the multi-way valve (13), another side of the plunger airway (10) is connected to the syringe plunger (6); a side of the pressure rod airway (15) is connected to a pressure rod mouth (16) of the multi-way valve (13), and another side of the pressure rod airway (15) is connected to the sealing pressure rod (9).

6. The electro-pneumatic intelligent syringe as claimed in claim 1, wherein the controller (19) is a system on a chip (SoC) microcontroller including a built-in memory, a central processing unit (CPU), interfaces detection and driving circuits, a wireless communication module, a wireless charging circuit, a BLUETOOTH module.

7. The electro-pneumatic intelligent syringe as claimed in claim 1, wherein a material of the component framework (18) is a plastic frame with and non-toxic.

8. The electro-pneumatic intelligent syringe as claimed in claim 2, wherein the needle sealing door (3) is a BLUETOOTH controlled electromagnetic sealing door, the BLUETOOTH controlled electromagnetic sealing door comprises an electromagnetic push rod (12) and a micro sealing door made of a non-toxic and wear-resistant plastic.

9. The electro-pneumatic intelligent syringe as claimed in claim 1, wherein the sealing stack (8) is a wear-resistant and non-toxic plastic sheet group coated with a wear-resistant sealing flexible coating, and the sealing pressure rod (9) is a micro pneumatic curved non-toxic plastic rod fixed on a wall of the plunger airway (10).

10. The electro-pneumatic intelligent syringe as claimed in claim 1, wherein the central airway (11), the plunger airway (10) and the pressure rod airway (15) are composed of silica gel foldable tubes.

11. An electro-pneumatic intelligent syringe, comprising:
a syringe needle (1), defining a hole;
a needle tube (2), connected to the syringe needle (1); wherein the needle tube (2) defines a needle through hole (5) connected to the hole of the syringe needle (1);
a needle sealing door (3), disposed between the syringe needle (1) and the needle tube (2) and configured to block the needle through hole (5);
an induction coil (4), disposed between the syringe needle (1) and the needle tube (2), and electrically connected to the needle sealing door (3);
a syringe plunger (6), disposed in the needle tube (2);
a sealing stack (8), embedded into a middle of the syringe plunger (6);
a sealing pressure rod (9), disposed on a side of the sealing stack (8) facing away from the syringe needle (1);
a plunger airway (10), connected to the syringe plunger (6) and configured to drive the syringe plunger (6) to move in the needle tube (2);
a central airway (11), connected to the sealing stack (8), and configured to control opening or closing of the sealing stack (8);
a pressure rod airway (15), connected to the sealing pressure rod (9); wherein the pressure rod airway (15) is configured to drive the sealing pressure rod (9) to lock the sealing stack (8), and the sealing stack (8) is not opened in a locking state;
a multi-way valve (13), connected to the plunger airway (10), the central airway (11) and the pressure rod airway (15); and
a suction-supply fan (17), connected to the multi-way valve (13).
